(19) United States
(12) Patent Application Publication
(10) Pub. No.: US 2023/0235275 A1
(43) Pub. Date: Jul. 27, 2023

(54) TRANSPORT MEDIUM FOR MICROORGANISM

(71) Applicant: Delta Electronics Int'l (Singapore) Pte Ltd, Singapore (SG)

(72) Inventors: You Bin Lin, Singapore (SG); Jia Jun Lee, Singapore (SG); Weishi Zhang, Singapore (SG)

(21) Appl. No.: 18/098,254

(22) Filed: Jan. 18, 2023

(30) Foreign Application Priority Data
Jan. 25, 2022 (SG) 10202200751V

(51) Int. Cl. C12N 1/20

(52) U.S. Cl. CPC C12N 1/20; C12N 2500/12; C12N 2500/50; C12N 2500/60; C12N 2500/44

(57) ABSTRACT

A transport medium for a microorganism is provided. The transport medium includes at least one chaotropic substance, at least one acid, at least one buffer, at least one chelating agent, and at least one detergent. The transport medium is able to inactivate bacteria and viruses at the time of sample collection, stabilize and preserve microbial nucleic acids at ambient temperature for extended periods of time.

### CROSS-REFERENCE TO RELATED APPLICATION

This application claims the priority to Singapore Patent Application No. 10202200751V filed on Jan. 25, 2022. The entirety of the aforementioned patent application is incorporated herein by reference.

### FIELD OF THE INVENTION

The present disclosure relates to a transport medium for a microorganism, and more particularly to a transport medium for bacteria and viruses.

### BACKGROUND OF THE INVENTION

New and emerging infectious respiratory diseases represent a continuing public health threat worldwide. In the last decade, two major pandemics, the 2009 H1N1 swine influenza and 2019 COVID-19, swept across the continents causing millions of infected cases. The speed which these viruses spread across the world is largely attributed to the increasing movement of people around the world and high population densities in urban areas.

To stem the spread of these emerging diseases, pathogen detection and characterization needs to be faster and more robust. With the advent of molecular diagnostics, turnaround time and accuracy of diagnosis has improved. However, one of the bottlenecks that hinders the efficiency and speed of molecular diagnosis occurs at the first step, i.e., sample collection. Current biological sample collection kits present a few shortcomings which limits the full advantage of molecular diagnostic. First, nucleic acids, e.g., DNA and RNA, in the biological sample quickly degrade at ambient temperature. To ensure the success of analysis, the integrity of the nucleic acids must be maintained. Therefore, upon collecting the sample, it is stored and transported in refrigeration/freezer. Secondly, the need for cold chain logistic for the temperature sensitive samples means more equipment or infrastructure are needed, which contributes to a higher cost. Thirdly, the biological samples are often infectious (containing live bacteria, virus or other pathogens) and the sample collection kit may not inactivate the infectious agents (some sample collection kits are intentionally formulated to maintain the viability of the pathogens). Therefore, a collected sample is rendered unsafe for individuals involved in collection, transfer and testing. Also, the testing has to be carried out in a higher biosafety level containment, which incurs high cost for equipment and infrastructure. Additionally, the transport of an infectious sample is a logistical headache especially if done cross-border, which increases the expense and effort.

This leads to the introduction of new generation sample collection kits/reagents that are designed for a molecular diagnostic workflow. These kits claim to be able to: (1) inactivate pathogens at the point of collection, (2) stabilize and preserve the nucleic acids, e.g., DNA and RNA, and (3) be transported at ambient temperature while preserving the nucleic acids. With these benefits, the above-mentioned drawbacks will be non-existent. However, most sample collection mediums contain ethanol, which pose a concern since there are alcohol-shipping restrictions in many countries.

Therefore, there is a need of providing a microorganism transport medium to address the above issues encountered by the prior arts.

### SUMMARY OF THE INVENTION

An object of the present disclosure is to provide a transport medium for a microorganism to inactivate and lyse the microorganism, as such releasing the microorganism's nucleic acids, and store and preserve the released nucleic acids.

In accordance with an aspect of the present disclosure, a transport medium for a microorganism is provided. The transport medium includes at least one chaotropic substance, at least one acid, at least one buffer, at least one chelating agent, and at least one detergent.

In accordance with another aspect of the present disclosure, a transport medium for a microorganism is provided. The transport medium includes at least one chaotropic substance, at least one acid, at least one buffer, at least one chelating agent, at least one detergent, and at least one precipitating agent.

In an embodiment, the transport medium for the microorganism includes 1 M to 4 M guanidine thiocyanate (GuSCN); 1 mM to 100 mM TRIS; 10 mM to 50 mM ethylenediaminetetraacetic acid (EDTA); 10 mM to 50 mM sodium citrate; and 0.1% to 2% NP-40, wherein the transport medium is alcohol-free.

In an embodiment, the transport medium further includes an acid to adjust the pH of the transport medium to between 4 to 7.

In an embodiment, the acid includes HCl.

In an embodiment, the transport medium further includes a precipitating agent.

In an embodiment, the precipitating agent includes polyethylene glycol (PEG), and the transport medium includes 1% to 15% PEG.

In an embodiment, the microorganism includes bacteria and viruses.

In an embodiment, the transport medium is used for a swab specimen.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 shows the culture result of Pseudomonas aeruginosa;

FIG. 2 shows the culture result of Staphylococcus aureus;

FIG. 3 shows the culture result of Mycobacterium fortuitum;

FIG. 4 shows the stability analysis of HCoV 229E, HCoV 0C43, FluA H3N2, and SARS-CoV-2 viral RNA stored and preserved in the transport medium;

FIG. 5 shows the stability analysis of Staphylococcus aureus DNA stored and preserved in the transport medium;

FIGS. 6A and 6B show the TCID50 assay results for FluA H1N1 and HCoV 0C43, respectively;

FIG. 7 shows the PCR test result and average Ct values from the PCR test on Influenza A virus detection; and

FIGS. 8A and 8B show the shelf life study of the transport medium.

### DETAILED DESCRIPTION OF THE PREFERRED EMBODIMENT

The present invention will now be described more specifically with reference to the following embodiments. It is to be noted that the following descriptions of the embodiments of this invention are presented herein for purpose of illustration and description only; it is not intended to be exhaustive or to be limited to the precise form disclosed.

The present disclosure is to provide a transport medium which is able to inactivate bacteria and viruses at the time of sample collection, stabilize and preserve microbial nucleic acids at ambient temperature for extended periods of time and display a performance comparable to a commercial specimen collection kit/reagent.

In one aspect, the present disclosure provides a transport medium which releases nucleic acids and microorganisms from the swab samples into the transport medium. The term “nucleic acid” includes both ribonucleic acid (RNA) and deoxyribonucleic acid (DNA) and further includes RNA and/or DNA which is linear or branched, single or double stranded or fragments thereof. The term “microorganism” refers to bacteria and viruses.

In another aspect, the present disclosure provides a transport medium for preserving and stabilizing the nucleic acids released from the swab samples and the nucleic acids released from the microorganisms into the transport medium. The term “preserved” refers to the nucleic acids which are protected from degradation and thereafter successfully isolated and analyzed using molecular biology methods.

When the swab samples are treated with the transport medium disclosed herein, the released nucleic acids from the swab samples and the nucleic acids released from the microorganism into the transport medium are preserved and stabilized whereby the nucleic acids are protected from degradation and can be later isolated from the sample and analyzed using molecular biology methods. The released nucleic acids preserved using the transport medium of the present disclosure can be isolated following extended periods of storage over a range of temperatures and can be used in molecular diagnostic applications.

Using the disclosed transport medium, the preservation of the nucleic acids can be over one year at ambient temperature.

The transport medium disclosed herein is also formulated for the lysis of microorganisms that may be present in the swab sample thereby reducing the health and safety risks associated with handling, transporting and testing of the treated swab sample.

The transport medium disclosed herein is an alcohol-free transport medium, thereby this allows the transport medium to bypass the alcohol-shipping restrictions in many countries.

The transport medium of the present disclosure is described in detail as follows.

According to a first illustrative example of the present disclosure, the transport medium includes at least one chaotropic substance, at least one acid, at least one buffer, at least one chelating agent, at least one detergent, and at least one precipitating agent.

In an embodiment, the chaotropic substance is but not limited to guanidine thiocyanate (GuSCN), and the transport medium may include from about 1 M to about 4 M GuSCN.

In an embodiment, the acid is but not limited to HCl. HCl is added to the transport medium to adjust the pH to between 4 to 7.

In an embodiment, the buffer is but not limited to TRIS, and the transport medium may include from about 1 mM to about 100 mM TRIS.

In an embodiment, the chelating agent is but not limited to ethylenediaminetetraacetic acid (EDTA), and the transport medium may include from about 10 mM to about 50 mM EDTA.

In an embodiment, the chelating agent is but not limited to sodium citrate (Na Citrate), and the transport medium may include from about 10 mM to about 50 mM Na Citrate.

In an embodiment, the detergent is but not limited to NP-40, and the transport medium may include from about 0.1% to about 2% NP-40.

In an embodiment, the precipitating agent is but not limited to polyethylene glycol (PEG), for example PEG 8000, and the transport medium may include from about 1% to about 15% PEG.

For example, the transport medium in Example 1 includes 2-4 M GuSCN as the chaotropic substance, HCl as the acid being added to adjust pH to between 5 to 7, 50-100 mM TRIS as the buffer, 20-30 mM EDTA and 20-30 mM Na Citrate as the chelating agents, 1-2% (wt/vol) NP-40 as the detergent, and 5-15% (wt/vol) PEG 8000 as the precipitating agent.

For example, the transport medium in Example 2 includes 2-4 M GuSCN as the chaotropic substance, HCl as the acid being added to adjust pH to between 5 to 7, 1-10 mM TRIS as the buffer, 20-30 mM EDTA and 20-30 mM Na Citrate as the chelating agents, 1-2% (wt/vol) NP-40 as the detergent, and 5-15% (wt/vol) PEG 8000 as the precipitating agent.

For example, the transport medium in Example 3 includes 2-3 M GuSCN as the chaotropic substance, HCl as the acid being added to adjust pH to between 6 to 7, 1-10 mM TRIS as the buffer, 10-20 mM EDTA and 25-50 mM Na Citrate as the chelating agents, 0.5-1% (wt/vol) NP-40 as the detergent, and 5-10% (wt/vol) PEG 8000 as the precipitating agent.

For example, the transport medium in Example 4 includes 2-3 M GuSCN as the chaotropic substance, HCl as the acid being added to adjust pH to between 6 to 7, 50-100 mM TRIS as the buffer, 10-20 mM EDTA and 25-50 mM Na Citrate as the chelating agents, 0.5-1% (wt/vol) NP-40 as the detergent, and 5-10% (wt/vol) PEG 8000 as the precipitating agent.

For example, the transport medium in Example 5 includes 2-3 M GuSCN as the chaotropic substance, HCl as the acid being added to adjust pH to between 4 to 6, 1-10 mM TRIS as the buffer, 10-20 mM EDTA and 25-50 mM Na Citrate as the chelating agents, 0.5-1% (wt/vol) NP-40 as the detergent, and 5-10% (wt/vol) PEG 8000 as the precipitating agent.

For example, the transport medium in Example 6 includes 2-3 M GuSCN as the chaotropic substance, HCl as the acid being added to adjust pH to between 4 to 6, 50-100 mM TRIS as the buffer, 10-20 mM EDTA and 25-50 mM Na Citrate as the chelating agents, 0.5-1% (wt/vol) NP-40 as the detergent, and 5-10% (wt/vol) PEG 8000 as the precipitating agent.

For example, the transport medium in Example 7 includes 1-3 M GuSCN as the chaotropic substance, HCl as the acid being added to adjust pH to between 6 to 7, 1-10 mM TRIS as the buffer, 10-20 mM EDTA and 25-50 mM Na Citrate as the chelating agents, 0.5-1% (wt/vol) NP-40 as the detergent, and 1-10% (wt/vol) PEG 8000 as the precipitating agent.

For example, the transport medium in Example 8 includes 1-3 M GuSCN as the chaotropic substance, HCl as the acid being added to adjust pH to between 6 to 7, 50-100 mM TRIS as the buffer, 10-20 mM EDTA and 25-50 mM Na Citrate as the chelating agents, 0.5-1% (wt/vol) NP-40 as the detergent, and 1-10% (wt/vol) PEG 8000 as the precipitating agent.

For example, the transport medium in Example 9 includes 1-3 M GuSCN as the chaotropic substance, HCl as the acid being added to adjust pH to between 4 to 6, 1-10 mM TRIS as the buffer, 10-20 mM EDTA and 25-50 mM Na Citrate as the chelating agents, 0.5-1% (wt/vol) NP-40 as the detergent, and 1-10% (wt/vol) PEG 8000 as the precipitating agent.

For example, the transport medium in Example 10 includes 1-3 M GuSCN as the chaotropic substance, HCl as the acid being added to adjust pH to between 4 to 6, 50-100 mM TRIS as the buffer, 10-20 mM EDTA and 25-50 mM Na Citrate as the chelating agents, 0.5-1% (wt/vol) NP-40 as the detergent, and 1-10% (wt/vol) PEG 8000 as the precipitating agent.

For example, the transport medium in Example 11 includes 3-4 M GuSCN as the chaotropic substance, HCl as the acid being added to adjust pH to between 6 to 7, 1-10 mM TRIS as the buffer, 10-20 mM EDTA and 20-30 mM Na Citrate as the chelating agents, 0.5-1% (wt/vol) NP-40 as the detergent, and 10-15% (wt/vol) PEG 8000 as the precipitating agent.

For example, the transport medium in Example 12 includes 3-4 M GuSCN as the chaotropic substance, HCl as the acid being added to adjust pH to between 6 to 7, 50-100 mM TRIS as the buffer, 10-20 mM EDTA and 20-30 mM Na Citrate as the chelating agents, 0.5-1% (wt/vol) NP-40 as the detergent, and 10-15% (wt/vol) PEG 8000 as the precipitating agent.

For example, the transport medium in Example 13 includes 3-4 M GuSCN as the chaotropic substance, HCl as the acid being added to adjust pH to between 4 to 6, 1-10 mM TRIS as the buffer, 10-20 mM EDTA and 20-30 mM Na Citrate as the chelating agents, 0.5-1% (wt/vol) NP-40 as the detergent, and 5-10% (wt/vol) PEG 8000 as the precipitating agent.

For example, the transport medium in Example 14 includes 3-4 M GuSCN as the chaotropic substance, HCl as the acid being added to adjust pH to between 4 to 6, 50-100 mM TRIS as the buffer, 10-20 mM EDTA and 20-30 mM Na Citrate as the chelating agents, 0.5-1% (wt/vol) NP-40 as the detergent, and 5-10% (wt/vol) PEG 8000 as the precipitating agent.

In one preferred embodiment (Embodiment One), the transport medium includes 3 M GuSCN as the chaotropic substance, HCl as the acid being added to adjust pH to 6 to 7 (e.g., pH 6.7), 10 mM TRIS as the buffer, 20 mM EDTA and 25 mM Na Citrate as the chelating agents, 1% (wt/vol) NP-40 as the detergent, and 10% (wt/vol) PEG 8000 as the precipitating agent.

It is noted that, the transport medium in the above Examples 1-14 and Embodiment One may include no alcohol. That is to say, the transport medium is an alcohol-free transport medium, which allows the transport medium to bypass the alcohol-shipping restrictions in many countries.

According to a second illustrative example of the present disclosure, the transport medium includes at least one chaotropic substance, at least one acid, at least one buffer, at least one chelating agent, and at least one detergent.

In an embodiment, the chaotropic substance is but not limited to guanidine thiocyanate (GuSCN), and the transport medium may include from about 1 M to about 4 M GuSCN.

In an embodiment, the acid is but not limited to HCl. HCl is added to the transport medium to adjust the pH to between 4 to 7.

In an embodiment, the buffer is but not limited to TRIS, and the transport medium may include from about 1 mM to about 100 mM TRIS.

In an embodiment, the chelating agent is but not limited to ethylenediaminetetraacetic acid (EDTA), and the transport medium may include from about 10 mM to about 50 mM EDTA.

In an embodiment, the chelating agent is but not limited to sodium citrate (Na Citrate), and the transport medium may include from about 10 mM to about 50 mM Na Citrate.

In an embodiment, the detergent is but not limited to NP-40, and the transport medium may include from about 0.1% to about 2% NP-40.

For example, the transport medium in Example 15 includes 2-4 M GuSCN as the chaotropic substance, HCl as the acid being added to adjust pH to between 5 to 7, 50-100 mM TRIS as the buffer, 20-30 mM EDTA and 20-30 mM Na Citrate as the chelating agents, and 1-2% (wt/vol) NP-40 as the detergent.

For example, the transport medium in Example 16 includes 2-4 M GuSCN as the chaotropic substance, HCl as the acid being added to adjust pH to between 5 to 7, 1-10 mM TRIS as the buffer, 20-30 mM EDTA and 20-30 mM Na Citrate as the chelating agents, and 1-2% (wt/vol) NP-40 as the detergent.

For example, the transport medium in Example 17 includes 2-3 M GuSCN as the chaotropic substance, HCl as the acid being added to adjust pH to between 6 to 7, 1-10 mM TRIS as the buffer, 10-20 mM EDTA and 25-50 mM Na Citrate as the chelating agents, and 0.5-1% (wt/vol) NP-40 as the detergent.

For example, the transport medium in Example 18 includes 2-3 M GuSCN as the chaotropic substance, HCl as the acid being added to adjust pH to between 6 to 7, 50-100 mM TRIS as the buffer, 10-20 mM EDTA and 25-50 mM Na Citrate as the chelating agents, and 0.5-1% (wt/vol) NP-40 as the detergent.

For example, the transport medium in Example 19 includes 2-3 M GuSCN as the chaotropic substance, HCl as the acid being added to adjust pH to between 4 to 6, 1-10 mM TRIS as the buffer, 10-20 mM EDTA and 25-50 mM Na Citrate as the chelating agents, and 0.5-1% (wt/vol) NP-40 as the detergent.

For example, the transport medium in Example 20 includes 2-3 M GuSCN as the chaotropic substance, HCl as the acid being added to adjust pH to between 4 to 6, 50-100 mM TRIS as the buffer, 10-20 mM EDTA and 25-50 mM Na Citrate as the chelating agents, and 0.5-1% (wt/vol) NP-40 as the detergent.

For example, the transport medium in Example 21 includes 1-3 M GuSCN as the chaotropic substance, HCl as the acid being added to adjust pH to between 6 to 7, 1-10 mM TRIS as the buffer, 10-20 mM EDTA and 25-50 mM Na Citrate as the chelating agents, and 0.5-1% (wt/vol) NP-40 as the detergent.

For example, the transport medium in Example 22 includes 1-3 M GuSCN as the chaotropic substance, HCl as the acid being added to adjust pH to between 6 to 7, 50-100 mM TRIS as the buffer, 10-20 mM EDTA and 25-50 mM Na Citrate as the chelating agents, and 0.5-1% (wt/vol) NP-40 as the detergent.

For example, the transport medium in Example 23 includes 1-3 M GuSCN as the chaotropic substance, HCl as the acid being added to adjust pH to between 4 to 6, 1-10 mM TRIS as the buffer, 10-20 mM EDTA and 25-50 mM Na Citrate as the chelating agents, and 0.5-1% (wt/vol) NP-40 as the detergent.

For example, the transport medium in Example 24 includes 1-3 M GuSCN as the chaotropic substance, HCl as the acid being added to adjust pH to between 4 to 6, 50-100 mM TRIS as the buffer, 10-20 mM EDTA and 25-50 mM Na Citrate as the chelating agents, and 0.5-1% (wt/vol) NP-40 as the detergent.

For example, the transport medium in Example 25 includes 3-4 M GuSCN as the chaotropic substance, HCl as the acid being added to adjust pH to between 6 to 7, 1-10 mM TRIS as the buffer, 10-20 mM EDTA and 20-30 mM Na Citrate as the chelating agents, and 0.5-1% (wt/vol) NP-40 as the detergent.

For example, the transport medium in Example 26 includes 3-4 M GuSCN as the chaotropic substance, HCl as the acid being added to adjust pH to between 6 to 7, 50-100 mM TRIS as the buffer, 10-20 mM EDTA and 20-30 mM Na Citrate as the chelating agents, and 0.5-1% (wt/vol) NP-40 as the detergent.

For example, the transport medium in Example 27 includes 3-4 M GuSCN as the chaotropic substance, HCl as the acid being added to adjust pH to between 4 to 6, 1-10 mM TRIS as the buffer, 10-20 mM EDTA and 20-30 mM Na Citrate as the chelating agents, and 0.5-1% (wt/vol) NP-40 as the detergent.

For example, the transport medium in Example 28 includes 3-4 M GuSCN as the chaotropic substance, HCl as the acid being added to adjust pH to between 4 to 6, 50-100 mM TRIS as the buffer, 10-20 mM EDTA and 20-30 mM Na Citrate as the chelating agents, and 0.5-1% (wt/vol) NP-40 as the detergent.

In another preferred embodiment (Embodiment Two), the transport medium includes 3 M GuSCN as the chaotropic substance, HCl as the acid being added to adjust pH to 6 to 7 (e.g., pH 6.7), 10 mM TRIS as the buffer, 20 mM EDTA and 25 mM Na Citrate as the chelating agents, and 1% (wt/vol) NP-40 as the detergent.

It is noted that, the transport medium in the above Examples 15-28 and Embodiment Two may include no alcohol. That is to say, the transport medium is an alcohol-free transport medium, which allows the transport medium to bypass the alcohol-shipping restrictions in many countries.

The transport medium of the present disclosure illustrated above provides a unique collection and preservation formulation, functioning as the transport medium, which releases nucleic acids and microorganisms from the swab samples into the transport medium.

The transport medium of the present disclosure illustrated above can be used for preserving and stabilizing the nucleic acids released from the swab samples and the nucleic acids released from the microorganisms into the transport medium.

The transport medium of the present disclosure illustrated above provides a collection and preservation formulation to inactivate and lyse microorganisms present in the biological swab specimen, as such releasing the microorganism's nucleic acids, and preserve the released nucleic acids within the biological swab specimen, all in a single reaction vessel. The released nucleic acids are stabilized whereby the nucleic acids are protected from degradation and can be later isolated from the sample and available for molecular diagnostic analysis. In addition, the formulation of the disclosed transport medium can enable the released nucleic acids to remain at least substantially stable at ambient temperature, without requiring consistent and constant cooler temperatures for storage, such as refrigeration or freezing.

The transport medium of the present disclosure illustrated above is ideal for clinical, field and deployment use, or for high volume sample collection/extraction. Swab specimens collected in both embodiments with the above-mentioned disclosed compositions are biologically inactivated, and may be safely shipped, typically even without refrigeration or dry ice.

The following are experiments illustrating the use of the transport medium of the present disclosure.

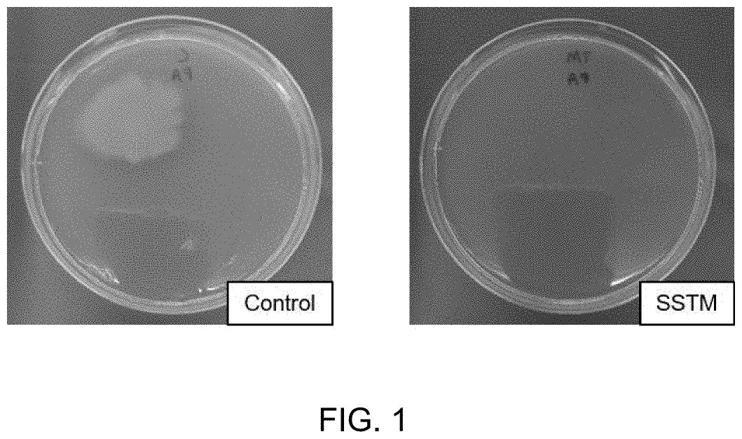

In the first experiment, the transport medium of the present disclosure was used for inactivation of gram-negative bacteria, Pseudomonas aeruginosa. Nasopharyngeal swab simulated matrix (NPSSM) was prepared as follows: NPSSM formula—1× PBS, 2.5% (w/v) Porcine mucin, 1% (v/v) Human whole blood, 0.85% (w/v) NaCl, 15% (v/v) Glycerol. 1×10⁶ Pseudomonas aeruginosa was spiked into 100 μl of NPSSM and 1.2 ml of the transport medium (prepared according to Embodiment Two's formulation, for example) was added into the spiked NPSSM and mixed well in an Eppendorf tube. After 10 minutes at room temperature (25° C.), 10 μl of treated spiked NPSSM was removed, diluted with 90 μl of tryptic soy buffer (TSB) (1:10 dilution) and plated on tryptic soy agar plate (TSAP). For control, 1.2 ml of PBS, instead of the transport medium, was added to the spiked NPSSM, mixed and let stand for 10 minutes, before plating as above-mentioned. The plates were incubated over 96 hours at 37° C. and observed for colonies. FIG. 1 shows the culture result of Pseudomonas aeruginosa. For the spiked NPSSM, no bacterial growth was observed on the plates that had been treated with the swab specimen transport medium (labeled ‘SSTM’), indicating that the bacterial cells were lysed. This confirms that the swab sample treated with the transport medium of the present disclosure was free of infectious gram-negative bacteria.

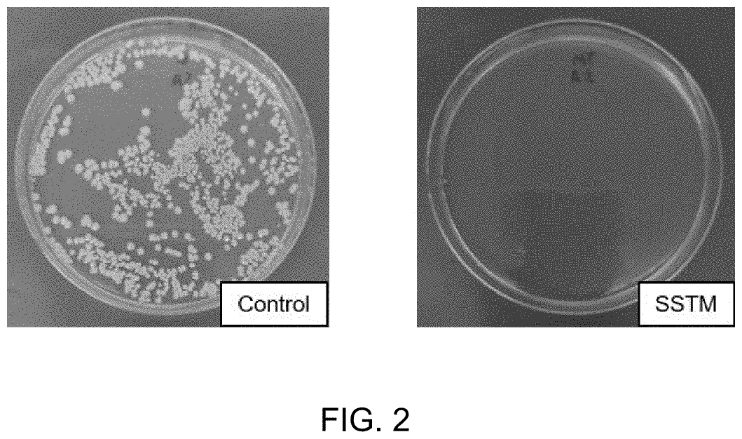

In the second experiment, the transport medium of the present disclosure was used for inactivation of gram-positive bacteria, Staphylococcus aureus. Nasopharyngeal swab simulated matrix (NPSSM) was prepared as follows: NPSSM formula—1× PBS, 2.5% (w/v) Porcine mucin, 1% (v/v) Human whole blood, 0.85% (w/v) NaCl, 15% (v/v) Glycerol. 1×10⁶ Staphylococcus aureus was spiked into 100 μl of NPSSM and 1.2 ml of the transport medium was added into the spiked NPSSM and mixed well in an Eppendorf tube. After 10 minutes at room temperature (25° C.), 10 μl of treated spiked NPSSM was removed, diluted with 90 μl of TSB (1:10 dilution) and plated on tryptic soy agar plate (TSAP). For control, 1.2 ml of PBS, instead of the transport medium, was added to the spiked NPSSM, mixed and let stand for 10 minutes, before plating as above-mentioned. The plates were incubated over 96 hours at 37° C. and observed for colonies. FIG. 2 shows the culture result of Staphylococcus aureus. For the spiked NPSSM, no bacterial growth was observed on the plates that had been treated with the swab specimen transport medium (labeled ‘SSTM’), indicating that the bacterial cells were lysed. This confirms that the swab sample treated with the transport medium of the present disclosure was free of infectious gram-positive bacteria.

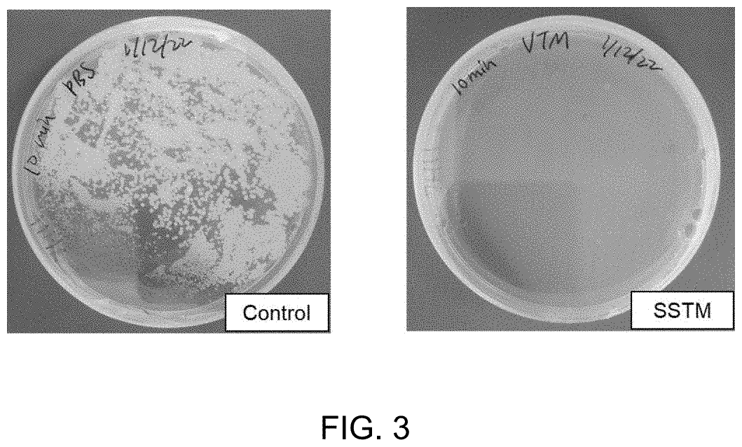

In the third experiment, the transport medium of the present disclosure was used for inactivation of mycobacteria, Mycobacterium fortuitum. Nasopharyngeal swab simulated matrix (NPSSM) was prepared as follows: NPSSM formula—1× PBS, 2.5% (w/v) Porcine mucin, 1% (v/v) Human whole blood, 0.85% (w/v) NaCl, 15% (v/v) Glycerol. 1×10⁶ Mycobacterium fortuitum was spiked into 100 μl of NPSSM and 1.2 ml of the transport medium was added into the spiked NPSSM and mixed well in an Eppendorf tube. After 10 minutes at room temperature (25° C.), 10 μl of treated spiked NPSSM was removed, diluted with 90 μl of PBS (1:10 dilution) and plated on Middlebrook 7H9 plate. For control, 1.2 ml of PBS, instead of the transport medium, was added to the spiked NPSSM, mixed and let stand for 10 minutes, before plating as above-mentioned. The plates were incubated over 4 days at 33° C. and observed for colonies. FIG. 3 shows the culture result of Mycobacterium fortuitum. For the spiked NPSSM, no bacterial growth was observed on the plates that had been treated with the swab specimen transport medium (labeled ‘SSTM’), indicating that the bacterial cells were lysed. This confirms that the swab sample treated with the transport medium of the present disclosure was free of infectious mycobacteria.

According to the above three experiments, the transport medium of the present disclosure has the capability to inactivate gram-negative bacteria, gram-positive bacteria, and mycobacteria within 10 minutes, and this strongly suggests that the transport medium of the present disclosure will be able to inactivate virus within the same time (i.e., 10 minutes). As such, a swab sample treated with the transport medium of the present disclosure is free of infectious bacteria and viruses.

In the fourth experiment, the transport medium of the present disclosure was used for storage and preservation of viral RNA, including viral RNA from Human Coronavirus (HCoV) 229E, HCoV 0C43, Influenza A virus (FluA) H3N2, and SARS-CoV-2. Live virus was spiked into 100 μl of NPSSM to a final concentration of 10000 pfu/ml, added immediately into 900 μl of the transport medium in an Eppendorf tube and mixed well. The treated spiked NPSSM was then stored at room temperature (27° C.). 200 μl of the treated spiked NPSSM was removed at the following time points: 0 week, 4 weeks, 6 weeks, 8 weeks, 10 weeks, and 12 weeks. At every time point, the RNA was isolated from the treated spiked NPSSM using a commercial kit (e.g., Qiagen Viral RNA Kit, Cat #52906). From the 60 μl of eluted purified RNA, 5 μl of RNA was used in a 20 μl total reaction using a commercial kit (e.g., KAPA SYBR® FAST One-Step qRT-PCR Master Mix (2×) Kit, Cat #KR0393) for qRT-PCR. The reaction setup is as follows:

| | Volume of component | Concentration |
|---|---|---|
| qRT-PCR components | per reaction (μl) | per reaction |
| qPCR Master Mix (2X) | 10 | 1X |
| KAPA RT Mix (50X) | 0.4 | 1X |
| RNase Free dH₂O | 3.8 | — |
| Forward primer (10 μM) | 0.4 | 200 nM |
| Reverse primer (10 μM) | 0.4 | 200 nM |
| Purified RNA | 5 | — |
| | Total volume: 20 μl | |

The RT-PCR program is as follows:

| | Step | Time | | Temperature |
|---|---|---|---|---|
| | Reverse Transcription | 5 | min | 42° C. |
| | Enzyme Activation | 3 | min | 95° C. |
| Cycling x40 | Denaturation | 10 | sec | 95° C. |
| | Annealing/Extension | 30 | sec | 60° C. |

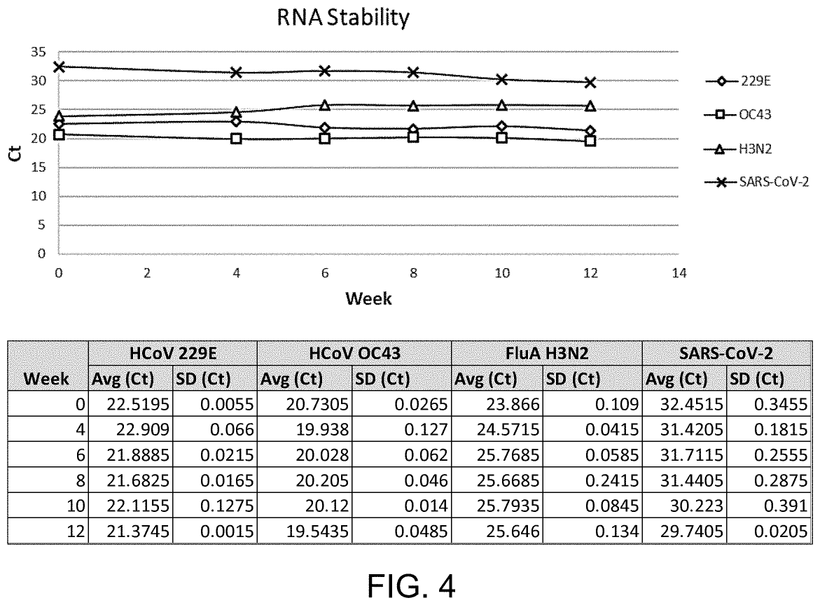

FIG. 4 shows the stability analysis of HCoV 229E, HCoV 0C43, FluA H3N2, and SARS-CoV-2 viral RNA stored and preserved in the transport medium. The successful amplification and detection of viral RNA was observed after 0 weeks, 4 weeks, 6 weeks, 8 weeks, 10 weeks, and 12 weeks of storage at room temperature in the transport medium. As the RNA is still detectable, this indicates the viral RNA remains intact after 12 weeks of storage at room temperature following collection. In other words, the viral RNA stays stable in the transport medium at room temperature over 12 weeks of incubation.

In the fifth experiment, the transport medium of the present disclosure was used for storage and preservation of bacterial DNA. 1×10⁵ CFU of live Staphylococcus aureus was spiked into 100 μl of NPSSM, added immediately into 900 μl of the transport medium in an Eppendorf tube and mixed well. The treated spiked NPSSM was then stored at room temperature (27° C.). 200 μl of the treated spiked NPSSM was removed at one month interval. At every time point, the DNA was isolated from the treated spiked NPSSM using a commercial kit (e.g., Qiagen QIAamp DNA Mini Kit, Cat #51306). From the 60 μl of eluted purified DNA, 5 μl of DNA was used in a 20 μl total reaction using a commercial kit (e.g., KAPA SYBR® FAST qPCR Kit Master Mix (2×) Universal, Cat #KK4602). The reaction setup is as follows:

| | Volume of component | Concentration per |
|---|---|---|
| qPCR components | per reaction (μl) | reaction |
| qPCR Master Mix (2X) | 10 | 1X |
| RNase Free dH₂O | 4.2 | — |
| Forward primer (10 μM) | 0.4 | 500 nM |
| Reverse primer (10 μM) | 0.4 | 500 nM |
| Purified DNA | 5 | — |
| | Total volume: 20 μl | |

The PCR program is as follows:

| | Step | Time | | Temperature |
|---|---|---|---|---|
| | Initial Denaturation | 3 | min | 95° C. |
| Cycling x40 | Denaturation | 3 | sec | 95° C. |
| | Annealing/Extension | 20 | sec | 60° C. |

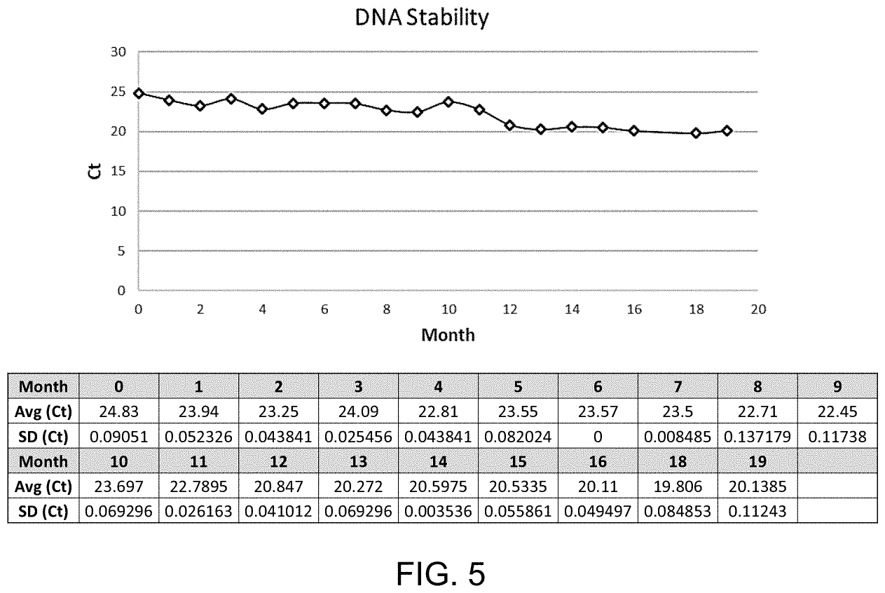

FIG. 5 shows the stability analysis of Staphylococcus aureus DNA stored and preserved in the transport medium. The successful amplification and detection of bacterial DNA was observed after 19 months of storage at room temperature in the transport medium. As the DNA is still detectable, this indicates the bacterial DNA remains intact after 19 months of storage at room temperature following collection. In other words, Staphylococcus aureus DNA stays stable in the transport medium at room temperature over 19 months of incubation.

In the sixth experiment, the transport medium of the present disclosure was used for inactivation of RNA viruses, including Influenza A virus and Human Coronavirus. 100 μl of 10⁵ TCID50/ml of live viruses FluA H1N1 and HCoV OC43 was respectively spiked into 300 μl of the transport medium. After 10 seconds incubation at room temperature (25° C.), the solution was processed with a detergent removal spin column to remove the cytotoxic effect. For control, 300 μl of PBS, instead of the transport medium, was added with the virus, mixed and let stand for 10 seconds, before processing as above-mentioned. TCID50 assay was performed by serial dilution of the processed elute with cell culture medium from 10\hat{ }0 to 10\hat{ }−5. The processed elute was used to infect live cell culture, incubated over 6 days at 37° C. and observed for cytopathic effect (CPE).

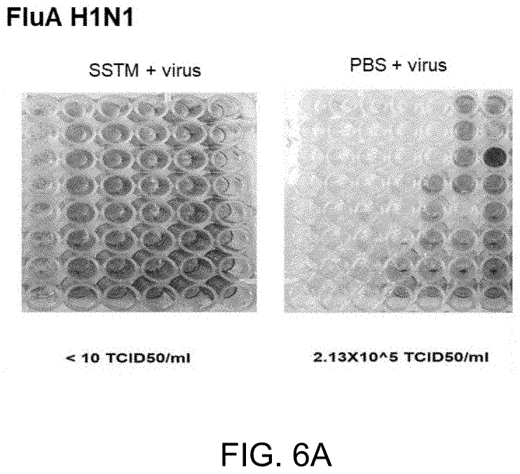

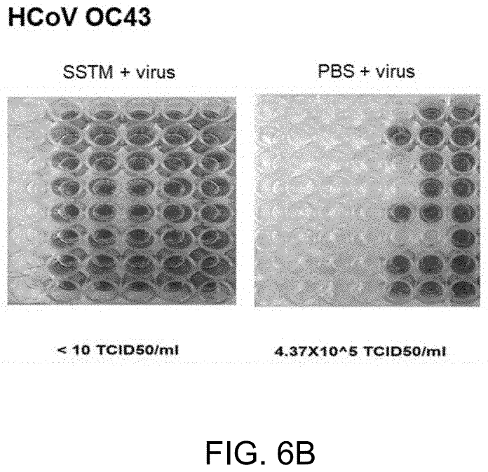

FIGS. 6A and 6B show the TCID50 assay results for FluA H1N1 and HCoV 0C43, respectively, wherein the virus infected wells were colorless and wells with live cells were stained in blue. For the solution containing the transport medium (labeled ‘SSTM’) and the live virus, the cells in serial dilution columns were alive, which means the transport medium successfully inactivated the virus. The results show that a 10⁴ TCID50/ml reduction of virus concentration was observed for both FluA H1N1 and HCoV OC43, indicating that the transport medium has virucidal activity.

In the seventh experiment, the recovery of RNA from low concentrations of organisms from the transport medium of the present disclosure was determined. The Influenza A Wuhan strain was spiked into nasopharyngeal swab simulated matrix (NPSSM) with final concentrations ranging from 10\hat{ }2 to 10\hat{ }4 pfu/ml. Aliquots of spiked NPSSM were prepared and added into the transport medium followed by processing using a commercial kit (e.g., Tiangen DP315-T8) for nucleic acid extraction. The reaction setup and RT-PCR program were the same as in the fourth experiment.

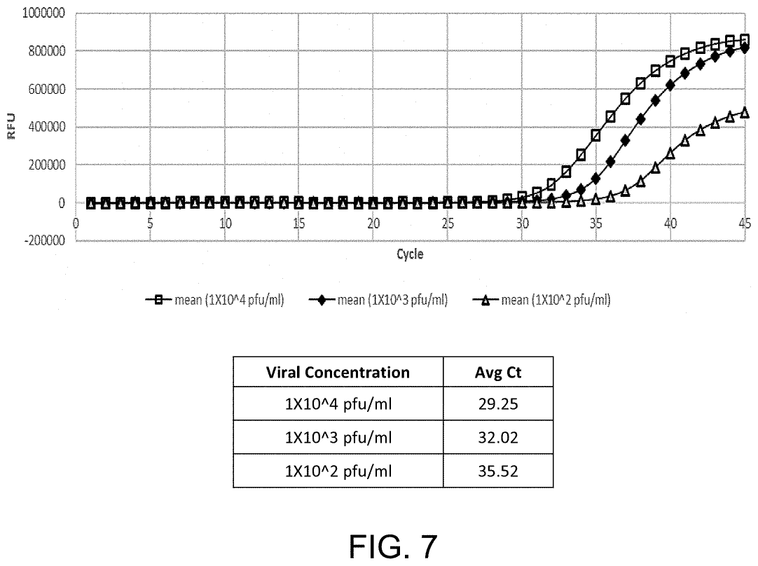

FIG. 7 shows the PCR test result and average Ct values from the PCR test on Influenza A virus detection. The Influenza A virus at different low concentrations in the transport medium were extracted with commercial nucleic acid extraction kit and followed by PCR assay with Influenza A detection. More specifically, the Influenza A virus in the transport medium at concentrations of 1×10\hat{ }4 pfu/ml to 1×10\hat{ }2 pfu/ml were extracted, and the extracted RNA was further amplified and detected with standard real-time RT-PCR assay. All concentrations including low concentration of 100 pfu/ml were consistently detectable, indicating that the transport medium is able to retain the downstream PCR assay sensitivity. The data demonstrated that the transport medium retained good assay sensitivity (100 pfu/ml) for downstream PCR applications.

In the eighth experiment, the shelf life of the transport medium of the present disclosure was determined. In the test, the transport medium was kept in a polypropylene plastic tube and stored at room temperature (27° C.), and an aliquot was taken out for the assay at each time point. 100 cfu/ml of live bacteria or 1000 pfu/ml of live virus was spiked into 50 μl of NPSSM, added immediately into 450 μl of the transport medium in an Eppendorf tube and mixed well. DNA from bacteria was isolated and analyzed according to the fifth experiment. RNA from virus was isolated and analyzed according to the fourth experiment.

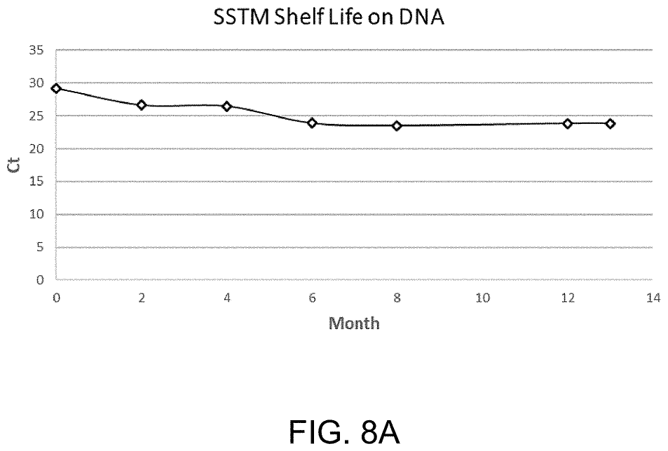

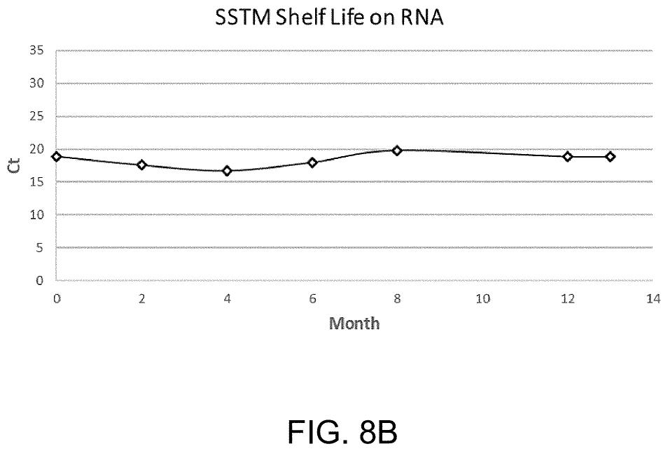

FIGS. 8A and 8B show the shelf life study of the transport medium. It is clear that the shelf life of the transport medium of the present disclosure is at least 13 months, and the transport medium of the present disclosure is able to retain its inactivation properties for DNA and RNA for a period of 13 months.

From the above, the present disclosure provides the transport medium to inactivate and lyse microorganisms present in the biological swab specimen, as such releasing the microorganism's nucleic acids, and store and preserve the released nucleic acids within the biological swab specimen, all in a single reaction vessel. The released nucleic acids are stabilized whereby the nucleic acids are protected from degradation and can be later isolated from the sample and available for molecular diagnostic analysis. In addition, the transport medium of the present disclosure can enable the released nucleic acids to remain at least substantially stable at ambient temperature, without requiring consistent and constant cooler temperatures for storage, such as refrigeration or freezing. It is also compatible with commercial nucleic acid extraction kits, retaining good sensitivity (100 pfu/ml) for downstream application. Further, the transport medium of the present disclosure is an alcohol-free transport medium, thereby this allows the transport medium to bypass the alcohol-shipping restrictions in many countries. Therefore, the transport medium of the present disclosure is ideal for clinical, field and deployment use.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A transport medium for a microorganism, comprising:
1 M to 4 M guanidine thiocyanate (GuSCN);
1 mM to 100 mM TRIS;
10 mM to 50 mM ethylenediaminetetraacetic acid (EDTA);
10 mM to 50 mM sodium citrate; and
0.1% to 2% NP-40,
wherein the transport medium is alcohol-free.

2. The transport medium according to claim 1, further comprising an acid to adjust the pH of the transport medium to between 4 to 7.

3. The transport medium according to claim 2, wherein the acid comprises HCl.

4. The transport medium according to claim 1, further comprising a precipitating agent.

5. The transport medium according to claim 4, wherein the precipitating agent comprises polyethylene glycol (PEG)

6. The transport medium according to claim 5, wherein the transport medium comprises 1% to 15% PEG.

7. The transport medium according to claim 1, wherein the microorganism comprises bacteria and viruses.

8. The transport medium according to claim 1, wherein the transport medium is used for a swab specimen.
